(19) United States
(12) Patent Application Publication
(10) Pub. No.: US 2024/0260843 A1
(43) Pub. Date: Aug. 8, 2024

(54) SYSTEM AND METHOD FOR MONITORING HEALTH DATA FROM INDIVIDUALS IN REAL TIME

(71) Applicant: Family Mednet, Inc., Boca Raton, FL (US)

(72) Inventors: Craig Winograd, Boca Raton, FL (US); Peter Ward, Plantation, FL (US)

(21) Appl. No.: 18/433,921

(22) Filed: Feb. 6, 2024

Related U.S. Application Data
(60) Provisional application No. 63/443,583, filed on Feb. 6, 2023.

(51) Int. Cl. A61B 5/0205; A61B 5/00; A61B 5/01; A61B 5/021; A61B 5/024; A61B 5/145; A61B 5/20; G16H 10/60; G16H 80/00

(52) U.S. Cl. CPC A61B 5/02055; A61B 5/0006; A61B 5/0008; A61B 5/002; A61B 5/0022; A61B 5/4839; A61B 5/681; A61B 5/743; A61B 5/7435; G16H 10/60; G16H 80/00; A61B 5/01; A61B 5/021; A61B 5/024; A61B 5/14532; A61B 5/14542; A61B 5/20; A61B 5/445; A61B 2560/045

(57) ABSTRACT

A device-agnostic system and a method for monitoring in real time health data of an individual including a cloud based server connected to a network, a tangible, non-volatile memory, at least one medical device wirelessly connected to the network, the at least one medical device is configured to generate health data from the individual; and at least one user interface device connected to the network, and the server is configured to store the health data and to allow the at least one user interface device to view the health data in real time.

### FIELD OF THE INVENTION

The invention relates to a unique system and a method for monitoring health data from an individual from one or multiple devices made by one or multiple manufacturers and storing the health data securely in a database. The health data can be remotely monitored in real time.

### BACKGROUND OF THE INVENTION

There is a great need for a device-agnostic simplified system and method for remotely monitoring health data from individuals.

### SUMMARY OF THE INVENTION

An embodiment of the system facilitates independent living for individuals and peace of mind for their loved ones. Utilizing current and emerging technologies, the present invention provides family members and healthcare providers with instant remote access to a loved one's in-home medical results. Through the system, whenever an individual takes an in-home reading with their compatible Bluetooth enabled FDA-approved medical device, the system will capture that data and transmit it securely to a cloud server. From wherever they are, family members, caretakers and healthcare professionals can then log in and monitor the individual's readings for any irregularities or events of concern in real-time. If any irregularities occur with the readings outside of the standard readings, alerts can be sent out via SMS and emails.

The software platform works on a proprietary hardware gateway, or any Bluetooth-enabled computer, smart phone or tablet, and requires only minimal interaction from the client for effective performance.

The system communicates with FDA-approved medical devices that utilize currently established wireless solutions (i.e. 802.XX, Bluetooth, Zigbee and Z-Wave (currently Bluetooth)) including blood pressure cuffs, glucometers, body weight scales, oximeters, thermometers and wearables such as Apple and Garmin watches. In the near future, we will expand our monitoring offerings to additional options such as wireless motion detection (to track movement or lack thereof within the residence) and home climate tracking (to capture temperature, moisture in the bed and humidity levels in the bathroom indicative of whether a bath or shower has taken place).

The system is suitable for relatives and caregivers of seniors aging-at-home, along with individuals recovering-at-home and their caretakers and concerned family members.

The system can enhance care for a variety of additional markets, such as on-demand support for our courageous US military both active duty and veterans in their ongoing medical needs. For example, the upcoming brand extension VeteranMedNet.com will bring this additional market targeted services unique to the needs of veterans.

Physical distance should not hinder the ability to monitor the health and wellbeing of recovering-at-home and aging-at-home family members. The system can work directly with doctors/caregivers for patient intervention that will improve quality of life for all involved.

The system can include HIPAA Compliant Proprietary cloud-based client app and portal, a team of technology experts, wireless monitoring, health data storage, and/or minimal response time for customer support based on a 24/7 call center in the United States.

A preferred embodiment of the system is real time health monitoring of an individual. Family members, physicians, hospital staff and caregivers will be able to remotely monitor individuals in need of continuous care on demand with alerting if significant changes occur. The system can offer monitoring to other health-related areas such as juvenile diabetes, asthma and COPD.

The system can be utilized to monitor individuals including but not exclusively: individuals aging-in-place individuals recovering-in-place, juvenile diabetes, addicts in recovery, obesity, veterans, Medicare and Medicaid recipient, assisted living facilities, assisted living services, home automation market, baby boomers, AARP and other senior citizen organization members, physician patients, elder care specialists (including lawyers and visiting nurses), out-patient clinics (such as ambulatory surgery), and homecare service providers via partnerships.

The system is preferably built from the ground up as an agnostic independent Web/mobile based platform that utilizes wireless products from established medical device companies. The system can also include a downstream caregiver intervention that is not being offered at this time by any competitor. These home healthcare companies and physician practices can be a powerful and effective sales force for us in this white label phase.

In the event that an individual is using a glucometer or other medical device on a platform that is not supported by the present system one can easily adopt such a device into the platform, with a minimal learning curve and training can be provided.

An aim of the system is to support all medical wireless monitoring devices certified on our agnostic platform. The system utilizes proprietary software that supports a smartphone, tablet, smart watch or Family Med Net proprietary hardware gateway. Updates and diagnostics can be conducted remotely rather than requiring on-site visits.

The system preferably fulfills HIPPA compliance and retains health data secure including data transport.

Objectives of the invention and other objectives can be obtained by a system for monitoring in real time health data of an individual comprising:
a cloud-based server connected to a network, the cloud-based server comprising a tangible, non-volatile memory;
at least one medical device wirelessly connected to the network, wherein the at least one medical device is configured to generate health data from the individual, and the at least one medical device having a unique Bluetooth MAC address that is associated with the individual; and
at least one user interface device connected to a wireless network, wherein the server is configured to store the health data and to allow the at least one user interface device to view the health data in real time and associate the health data with the individual based on the unique MAC address of the Bluetooth device.

Objectives of the invention and other objectives can also be obtained by a method of monitoring health data of an individual in real time comprising providing the system; using the user interface device to remotely monitor and report changes in the health data of the individual in real time; and storing the health data in the tangible, non-volatile memory.

### BRIEF DESCRIPTION OF THE DRAWINGS

FIG. 1 illustrates an example of the system. This demonstrates the system and the embodiment of the invention. Various networks are implemented in accordance with embodiments of the invention, including a wired or wireless local area network (LAN) and a wide area network (WAN), wireless personal area network (PAN) and other types of networks including Bluetooth

FIG. 2 illustrates a flow chart example of the method and system. This demonstrates the collection of data from different devices from different manufacturers and sending the data in a unique fashion securely to server to create a historical snapshot overtime for a patient's health status giving loved ones and health care providers advance notice of potential issues.

FIG. 3 illustrates an example of a process flow for the method and system. This demonstrates the collection of data from different devices from different manufacturers and sending the data in a unique fashion securely to server to create a historical snapshot overtime for a patient's health status giving loved ones and health care providers advance notice of potential issues, in another graphical form.

FIG. 4 illustrates an example of the system monitoring in real time the health of soldiers. This demonstrates the collection of data from different devices from different manufacturers and sending the data in a unique fashion securely to server to create a historical snapshot overtime for soldiers' health status giving unit leaders and field hospitals advance notice of potential issues.

FIG. 5 illustrates an example of the system monitoring in real time the health of soldiers. This demonstrates the collection of data from different devices from different manufacturers as well as the ability to track a potential gunshot wound and sending the data in a unique fashion securely to server to create a historical snapshot overtime for soldiers health status giving unit leaders and field hospitals advance notice of potential issues.

FIG. 6 illustrates an example of the system monitoring in real time the health of soldiers. This demonstrates the collection of data from different devices from different manufacturers giving soldiers with Diabetes (previously not being able to serve due to this illness) and sending the data in a unique fashion securely to server to create a historical snapshot overtime for soldiers health status giving unit leaders and field hospitals advance notice of potential issues.

FIG. 7 illustrates an example of the system monitoring in real time the health of soldiers. This demonstrates the collection of data from a smart bandage and sending the data in a unique fashion securely to server to create a historical snapshot overtime for soldiers health status giving unit leaders and field hospitals advance notice of potential issues.

FIG. 8A is a screenshot of a mobile app client registration page. This APP Screen demonstrates the collection of data for a monitored patient/end user to register for the FamilyMedNet Platform. This information can either be entered through this secure APP or through a secure registration site.

FIG. 8B is a screenshot of a mobile app login page. This APP Screen demonstrates the login page for the APP (Same for Web Portal) for access to the FamilyMedNet Platform. We do provide another link to sign up.

FIG. 8C is a screenshot of a mobile app device dashboard. This APP screen demonstrates device platform for a monitored patient on the FamilyMedNet Platform. The patient, family member and healthcare professional can easily add on approved BlueTooth Devices (Even Apple Health). This is associated with the unique registration (Pairing) of the Bluetooth Devices MAC address for the patient.

FIG. 8D is a screenshot of a mobile app request for Bluetooth access. This APP screen demonstrates acknowledgment to use the registered devices to start taking reading from the Bluetooth device for a monitored patient on the FamilyMedNet Platform.

FIG. 8E is a screenshot of a mobile app Apple health kit access. This APP screen demonstrates the integration with Apple Health for a monitored patient's metrics on the FamilyMedNet Platform. This helps to expand the Apple Health reporting including SMS to healthcare professionals. Sharing of this data is unlimited.

FIG. 8F is a screenshot of a mobile app Apple health request to track health data. This APP screen demonstrates the integration with Apple Health for a monitored patient's metrics on the FamilyMedNet Platform. This helps to expand the Apple Health reporting including SMS to healthcare professionals. Sharing of this data is unlimited. You can toggle off and on the Health Care Access.

FIG. 8G is a screenshot of a mobile app device selection. This APP screen demonstrates device platform for a monitored patient on the FamilyMedNet Platform. Patient, family member and or healthcare Professional can take readings of several devices currently simply by tapping on a device image shown in this figure.

FIG. 8H is a screenshot of a mobile app multi-monitoring device connection. This APP screen demonstrates a supported multi-type device (Seen above the APP Screen) for a monitored patient on the FamilyMedNet Platform. Patient, family member and or healthcare Professional can take readings of several different currently simply by taping on a device show in this figure.

FIG. 8I is a screenshot of a mobile app displaying the daily recorded health data including oxygen, heart rate, PI (perfusion index) and BPI (blood pressure). This APP screen demonstrates the daily/weekly check on areas to be monitored thru the Multi-Type Device from Figure “8H” for a monitored patient on the FamilyMedNet Platform.

FIG. 8J is a screenshot of a mobile app displaying the daily recorded health data including oxygen, heart rate, PI (perfusion index) and BPI (blood pressure). This APP screen demonstrates the Pulse and Oximeter check for a monitored patient thru the Multi-Type Device from Figure “8H” for a monitored patient on the FamilyMedNet Platform. Notice the ‘Face” making it quicker to decect whether the reading was either favorable or not. These metrics can be set based on the patient's needs.

FIG. 8K is a screenshot of a mobile app displaying the daily recorded health data including temperature. This APP screen demonstrates the Thermometer check for a monitored patient thru the Multi-Type Device from Figure “8H” for a monitored patient on the FamilyMedNet Platform. Metrics can be set based on the patient's normal temperature for reporting.

FIG. 8L is a screenshot of a mobile app displaying Apple health and fitness kit connected to the system and real-time health data generated. This APP screen demonstrates the integration with Apple Health for a monitored patient's metrics on the FamilyMedNet Platform. This helps to expand Apple's Health reporting including SMS to healthcare professionals. Sharing of this data is unlimited. You can toggle off and on the Health Care Access. We have even incorporated steps, headphone audio levels. Imagine calling your parents to ask them why they are listening to music so loud. This could be an indication of hearing loss.

FIG. 8M is a screenshot of a mobile app displaying Apple health and fitness kit connected to the system and real-time health data generated. This APP screen demonstrates the integration with Apple Health for a monitored patient's metrics on the FamilyMedNet Platform. This helps to expand the Apple Health reporting on walking and exercise. This helps to expand the Apple Health reporting including SMS to family members and healthcare professionals. Sharing of this data is unlimited.

FIG. 8N is a screenshot of a mobile app displaying the Apple health and fitness kit dashboard. This APP screen demonstrates the integration with Apple Health for a monitored patient's metrics on the FamilyMedNet Platform. This helps to expand the Apple Health reporting on walking, swimming and exercise. This helps to expand the Apple Health reporting including SMS to family members and healthcare professionals. Sharing of this data is unlimited. This is great for someone in training.

FIG. 8O is a screenshot of a mobile app displaying Apple health and fitness kit connected to the system and real-time health data generated while running. This APP screen demonstrates the integration with Apple Health for a monitored patient's metrics on the FamilyMedNet Platform. This helps to expand the Apple Health reporting on Running on a daily basis This helps to expand the Apple Health reporting including SMS to family members and trainers. Sharing of this data is unlimited.

FIG. 8P is a screenshot of a mobile app displaying Apple health and fitness kit connected to the system and real-time health data generated while walking. This APP screen demonstrates the integration with Apple Health for a monitored patient's metrics on the FamilyMedNet Platform. This helps to expand the Apple Health reporting on walking on a daily basis. This helps to expand the Apple Health reporting including SMS to family members and trainers. Sharing of this data is unlimited.

FIG. 8Q is a screenshot of Apple watches connected to the system. This demonstrates the Apple Watchb screen with Apple Health for a monitored patient's metrics on the FamilyMedNet Platform. This step shows the data for the collection of the Blood Oxygen levels to be displayed in Figure “8R” APP Screen.

FIG. 8R is a screenshot of a mobile app displaying health data generated by the Apple watch. This APP screen demonstrates the integration with Apple Health for a monitored patient's metrics on the FamilyMedNet Platform. This helps to expand the Apple Health reporting on Blood Oxygen on a daily basis pushed from the Apple Watch in Figure “8Q”. This helps to expand the Apple Health reporting including SMS to family members and trainers. Sharing of this data is unlimited.

FIG. 8S is a screenshot of a mobile app displaying health data generated by the Apple watch including ECG. This APP screen demonstrates the integration with Apple Health for a monitored patient's metrics on the FamilyMedNet Platform. This helps to expand the Apple Health reporting on the ECG “Electrocardiogram” on a daily basis pushed from the Apple Watch in Figure “8Q”. This helps to expand the Apple Health reporting including SMS to family members and trainers. Sharing of this data is unlimited.

FIG. 8T is a screenshot of a mobile app displaying health data generated by the Apple watch including the oxygen level. This APP screen demonstrates the integration with Apple Health for a monitored patient's metrics on the FamilyMedNet Platform. This helps to expand the Apple Health reporting on the Oxygen Levels on a daily basis This helps to expand the Apple Health reporting including SMS to family members and trainers. Sharing of this data is unlimited.

FIG. 8U is a screenshot of displaying a Web Portal for a health provider. This webpage Login screen demonstrates the Healthcare provider login FamilyMedNet Platform. Notice the ability to monitor multiply patients.

FIG. 8V is a screenshot of a mobile app displaying a list of patients for the health provider. The following webpage screen demonstrates a HealthCare Professionals list of monitored patients portal on the FamilyMedNet Platform. This could be a White Label Platform where the Healthcare profession may Private Brand the pages for their practice.

FIG. 8X is a screenshot of a mobile app displaying patient details for the health provider. The following webpage screen demonstrates a patient status portal on the FamilyMedNet Platform. All the Patients metrics will reside on this screen like Blood Pressure, Oxygen Levels, Glucose Levels for example. A picture of the patient can be uploaded for instant recognition.

FIG. 8Y is a screenshot of a mobile app displaying a patient or family member information for the health provider. This webpage Login screen demonstrates the Family Member login for the FamilyMedNet Platform.

FIG. 8Z is a screenshot of a patient or family member's information for the health provider. This webpage screen demonstrates a parents status portal on the FamilyMedNet Platform. All the parents metrics will reside on this screen like Blood Pressure, Oxygen Levels, Glucose Levels for example. A picture of the patient can be uploaded for instant recognition if you are monitoring multiple parents.

FIG. 8AA is a screenshot of a mobile app displaying recorded health data for a patient.

### DETAILED DESCRIPTION OF THE INVENTION

In the following description, for purposes of explanation and not limitation, specific details are set forth, such as particular networks, communication systems, computers, terminals, devices, components, techniques, data and network protocols, software products and systems, operating systems, development interfaces, hardware, etc. in order to provide a thorough understanding of the present invention.

However, it will be apparent to one skilled in the art that the present invention can be practiced in other embodiments that depart from these specific details. Detailed descriptions of well-known networks, communication systems, computers, terminals, devices, components, techniques, data and network protocols, software products and systems, operating systems, development interfaces, and hardware are omitted so as not to obscure the description.

The method and system will now be explained with reference to the attached non-limiting drawings. The operations described in the figures and herein can be implemented as executable code stored on a computer or machine readable non-transitory tangible storage medium (e.g., floppy disk, hard disk, ROM, EEPROM, nonvolatile RAM, CD-ROM, etc.) that are completed based on execution of the code by a processor circuit implemented using one or more integrated circuits. The operations described herein also can be implemented as executable logic that is encoded in one or more non-transitory tangible media for execution (e.g., programmable logic arrays or devices, field programmable gate arrays, programmable array logic, application specific integrated circuits, etc.).

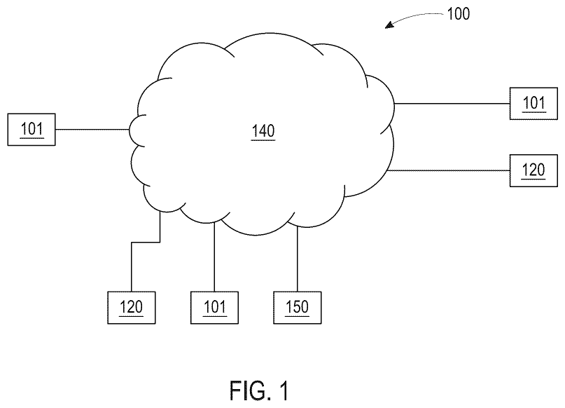

FIG. 1 illustrates a system 100 according to an embodiment of the invention. Various networks 140 may be implemented in accordance with embodiments of the invention, including a wired or wireless local area network (LAN) and a wide area network (WAN), wireless personal area network (PAN) and other types of networks. The network 140 includes connections over the Internet, an Intranet, Extranet, Ethernet, telephone network, or any other system that provides communications. When used in a LAN networking environment, computers may be connected to the LAN through a network interface or adapter. When used in a WAN networking environment, computers typically include a modem or other communication mechanism. Modems may be internal or external and may be connected to the system bus via the user-input interface, or other appropriate mechanism. Computers can be connected over the Internet, an Intranet, Extranet, Ethernet, telephone network, or any other system that provides communications. Some suitable communications protocols may include TCP/IP, UDP, OSI, Ethernet, WAP, IEEE 802.11, Bluetooth, Zigbee, Zwave or any other desired protocol. Furthermore, components of the system may communicate through a combination of wired or wireless paths.

The system 100 can be accessed via any user interface device 120 that is capable of connecting to the main server 150 via the network 140 or directly to the server 150 if desired. The user interface device 120 preferably comprises a keyboard or touch screen display to input information.

An exemplary user interface device 120 contains a web browser or similar program, allowing in some embodiments for a secure SSL connection, and able to display HTML and CSS. This includes user interface devices 120 such as tablets, iPads, Mac OS computers, Windows computers, e-readers, workstations, and mobile user devices such as Apple watch and other smart watches, the iphone, Android, Chrome Book and Windows devices (tablets). The user interface devices 120 can connect to the server via the internet and/or wirelessly, such as through a mobile telephone network 140, and/or any other suitable medium. User interface devices 120 are preferably able to communicate with the main server 150 so that content can be started on one user interface device 120 and later continued on a separate user interface device 120.

The main server 150 described herein can include one or more computer systems directly connected to one another and/or connected over the network 140. Preferably, the main server 150 is cloud-based. Each computer system can include a processor, tangible, non-volatile memory, user input and user output mechanisms, a network interface, and executable program code (software) comprising computer executable instructions stored in non-transitory tangible memory that executes to control the operation of the main server 150. Similarly, the processors functional components formed of one or more modules of program code executing on one or more computers. Various commercially available computer systems and operating system software can be used to implement the hardware and software. The components of each server can be co-located or distributed. In addition, all or portions of the same software and/or hardware can be used to implement two or more of the functional servers (or processors) shown. The main server 150 can run any desired operating system, such as Windows, Mac OS X, Linux, Unix, POSIX, Solaris or any other server based operating systems. Other embodiments can include different functional components. In addition, the present invention is not limited to a particular environment or main server 150 configuration. The main server 150 can be a cloud-based computer system.

Examples of non-volatile memory include flash memory, read-only memory (ROM), ferroelectric RAM, most types of magnetic computer storage devices (e.g. hard disk drives, floppy disks, and magnetic tape), optical discs, and early computer storage methods such as paper tape and punched cards.

The main server 150 preferably includes a web server and a query processing unit. The web server receives the user requests and sends it to the query processing unit. The query processing unit processes the request and responds back to the user interface device 120 via the web server. The query processing unit fetches data from the database server if additional information is needed for processing the request.

The main server 150 can include a plurality of individual computer systems directly connected and/or connected over network 140. Software program modules and data can be stored in the non-volatile memory of the main server 150 may be arranged in logical collections of related information on a plurality of computer systems having associated non-volatile memories. The main server 150 can comprise the non-volatile memory or the main server 150 can be in communication with the non-volatile memory storing the database.

The software (computer program code) required to operate the system 100 and health data can be stored in the non-volatile memory using any data structures known in the art including files, arrays, linked lists, relational database tables and the like. The stored data can include, for example, client information, and health data.

Medical devices 101 are connected to the network 140 via Bluetooth, or another protocol that provides an individual identification for each medical device 101. Bluetooth is preferred. Non-limiting examples of medical devices 101 include oxygen saturation, heart rate, blood pressure, activity, brain waves, blood glucose, body weight scales, oximeters, ECG, sleep monitors, thermometers and other new and emerging Bluetooth technologies. Bluetooth provides a unique MAC address for each medical device 101. Thus, the medical devices 101 for each individual will have associated MAC addresses. The system 100 utilizes the MAC address to properly associate the health data generated by the medical device 101 with the individual. As an example, a physician can use an IPad or Android Tablet to walk into a room and gather in real time health data from individuals and the system 100 will properly associate the health data with the correct individual using the unique MAC address for each medical device.

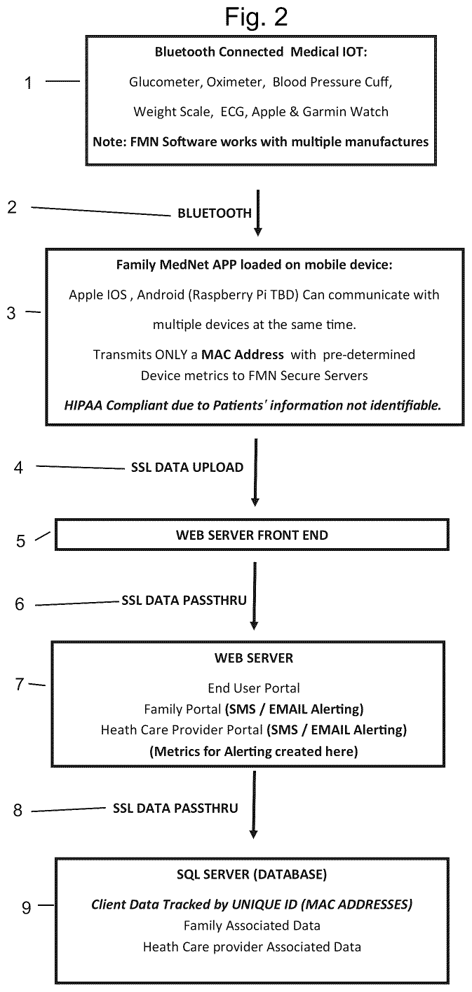

FIG. 2 The Family Mednet system provides a comprehensive platform for monitoring and managing health metrics using IoT devices, mobile applications, secure data transmission, and web-based portals. It facilitates proactive healthcare management by enabling real-time monitoring and timely intervention in case of abnormalities.
1. IoT Devices: Various IoT devices such as glucometers, blood pressure cuffs, weight scales, ECG monitors, Apple Watches, Garmin Watches, smart bandages, and other emerging IoT technologies are capable of transmitting monitored data over wireless Bluetooth. Each device is associated with a unique MAC Address, which serves as a primary identifier.
2. Bluetooth Wireless—Sending Metrics to APP.
3. Family Mednet App: The Family Mednet app can be installed on smart devices including Apple smartphones, Android-based smartphones, tablets (both Apple and Android), and Raspberry Pi devices. These devices utilize Bluetooth technology to receive data from the IoT devices.
4. Data Transmission: The data collected by the IoT devices is transmitted via SSL (Secure Sockets Layer) to the Front End Web Server(s). SSL ensures secure communication between the smart devices and the server.
5. Front End Web Server: The Front End Web Server(s) receives data from the smart devices running the Family Mednet app. This server likely manages the user interface and interactions for the Family Mednet portal.
6. Data Transmission: The data collected by the APP is transmitted via SSL (Secure Sockets Layer) to the Web Server(s). SSL ensures secure communications
7. Family Mednet Portal: (WebServer) Users, their families, and healthcare professionals have access to the Family Mednet Portal. This portal allows them to view both past and present health metrics monitored by the system. Abnormality Detection and Alerts: Users can set predetermined thresholds for health metrics. If an abnormality is detected, as defined by the user, alerts are sent via SMS (Short Message Service, text) and email to the user, their family members, and healthcare professionals.
8. Data Transmission: The data collected by the APP is transmitted via SSL (Secure Sockets Layer) to the Web Server(s). SSL ensures secure communications
9. Family Mednet SQL Server: The received data is securely stored in the Family Mednet SQL Server. The MAC Address is used to identify the device user's identity and securely place the device metrics in the user's space in the portal.

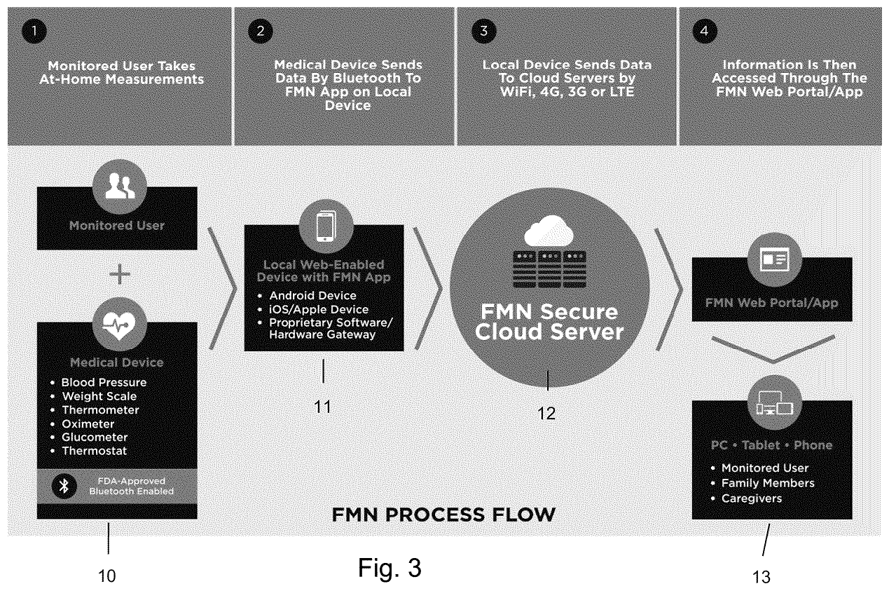

FIG. 3 The Family Mednet FMN Process Flow System provides a comprehensive platform for remote health monitoring, allowing users to track their health metrics conveniently from home or while traveling. It enables timely interventions and support from caregivers and healthcare professionals, enhancing overall healthcare management and quality of life for users.
10. Bluetooth Medical Devices: Users can utilize various FDA-approved Bluetooth-enabled medical devices such as glucometers, blood pressure cuffs, weight scales, oximeters, thermometers, home thermostats, and other approved devices to monitor their health metrics at home or while traveling.
11. Family Mednet (FMN) App: The FMN app is compatible with smart devices including iOS/Apple devices, Android-based devices, and proprietary software/hardware gateways like Raspberry Pi. These devices serve as the intermediary between the Bluetooth medical devices and the FMN system. Data Transmission: The Bluetooth medical devices communicate with the FMN app loaded on the smart device. The FMN app then forwards the monitored health statistics over Wi-Fi, 4G, 3G, or LTE to the Family Mednet secure cloud server.
12. Family Mednet Secure Cloud Server: The monitored health data is securely stored in the Family Mednet secure cloud server, ensuring privacy and confidentiality. Each user has their private health portal where their health metrics are stored. Accessing Information: Users, their families, and healthcare professionals can access the monitored user's health metrics through either the Family Mednet web portal or the FMN app. Multiple interfaces are available to cater to the specific needs of the monitored user, family member, or caregiver.
13. Monitoring and Care: The FMN (Web Portal/APP) system allows for remote monitoring and care management. Users can keep track of their health metrics, while family members and healthcare professionals can also access the data to provide necessary support and interventions as needed.

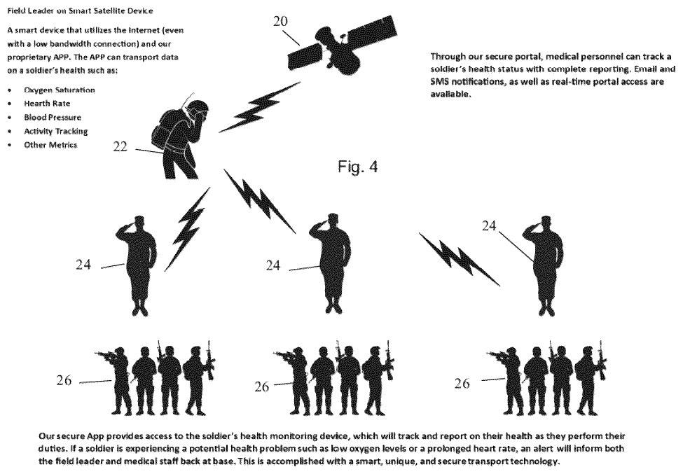

FIG. 4 shows The Family MedNet (FMN) technology presents a significant advancement in monitoring the health of soldiers on the battlefield. Here's how soldiers can benefit from this technology:
Real-Time Health Monitoring: Soldiers 26 are equipped with Bluetooth wearables such as smartwatches that monitor vital health metrics including blood pressure, oxygen saturation, heart rate, and activity tracking. These devices transmit real-time health data to their unit leader 24 via Bluetooth connectivity.
Secure Communication Infrastructure: The soldiers' communications soldier 22 carries an Android device with satellite internet capability, enabling secure communication with the military's private satellite network 20. The FMN app loaded on this device facilitates the transmission of health data to the FMN server on the military network.
Unit Leader's Role: The unit leader 24 carries a ruggedized Android tablet or similar device with the FMN app loaded on it. They receive health data from soldiers 26 and transmit it through the communications soldier 22 to the satellite network 20 for integration into the FMN server.
Alert System: The FMN app features an alert system that notifies both the unit leader and medical staff at the base in real-time if a soldier experiences potential health issues such as low oxygen levels or elevated heart rate. This timely alert enables swift response and appropriate medical intervention as needed.
Secure Transport Technologies: FMN utilizes smart and secure transport technologies to ensure the confidentiality and integrity of the transmitted health data. This ensures that sensitive health information remains protected throughout the transmission process.
In summary, the FMN technology provides soldiers with a safe and secure means to monitor their health in real-time while performing their duties on the battlefield. It enables proactive intervention and support from unit leaders and medical

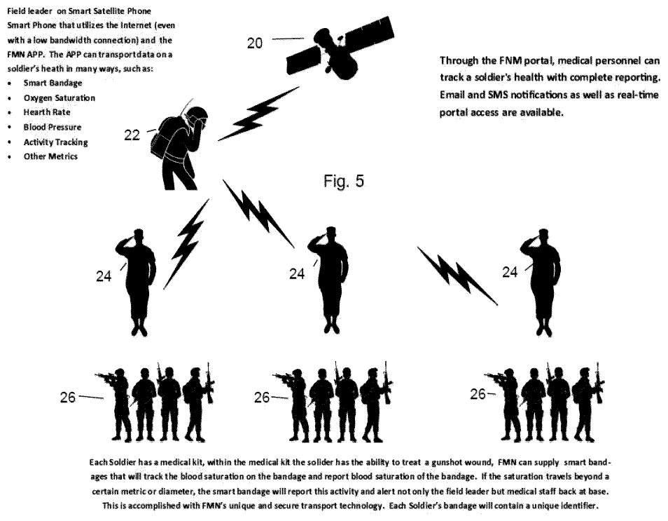

FIG. 5 The Family MedNet (FMN) technology offers critical benefits for soldiers, especially in the event of being wounded on the battlefield. Here's how soldiers can benefit from FMN technology after being wounded:
Real-Time Health Monitoring: 26 Soldiers wear Bluetooth-enabled smartwatches and other wearables that continuously monitor vital health metrics such as blood pressure, oxygen saturation, heart rate, and activity tracking. This data is transmitted in real-time to the unit leader 24 via Bluetooth connectivity.
Secure Communication Infrastructure: The communications soldier 22 carries an Android device with satellite internet capability to communicate with the military's private network 20. The FMN app loaded on this device enables secure transmission of health data to the FMN server on the military network.
Unit Leader's Role: The unit leader 24 carries a ruggedized Android tablet or similar device with the FMN app loaded on it. They receive health data from wounded soldiers 26 and transmit it through the communications soldier 22 to the satellite 20 for integration into the FMN server.
Monitoring Wound Status: The FMN app provides real-time access to the soldiers' health, including monitoring the status of gunshot wounds or other injuries. It tracks and reports the condition of a soldier's health as they perform their duties, even after being wounded.
Alert System for Critical Situations: If a soldier experiences potential health issues such as low oxygen levels, prolonged elevated heart rate, or excessive bleeding from a wound, the FMN app triggers alerts to both the unit leader and medical staff at the base in real-time. This allows for immediate intervention and medical assistance, potentially saving lives in critical situations.
Secure Transport Technologies: FMN utilizes smart and secure transport technologies to ensure the confidentiality and integrity of the transmitted health data, even in high-stress situations. This ensures that crucial health information remains protected throughout the transmission process.
In summary, FMN technology plays a crucial role in monitoring soldiers' health status, especially in the event of being wounded on the battlefield. It enables timely intervention and medical support, contributing to better outcomes and increased safety for soldiers in challenging environments.

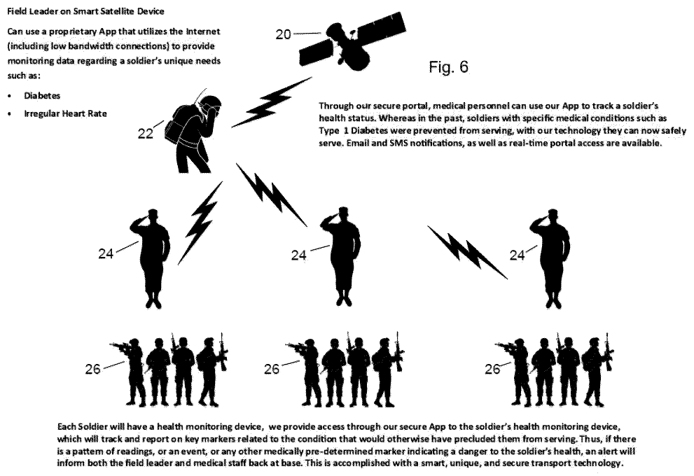

FIG. 6 The Family MedNet (FMN) technology offers significant benefits for potential soldiers with conditions like diabetes and irregular heart rates, enabling them to serve in the military while still being closely monitored for their health status. Here's how potential soldiers can benefit from FMN technology:
Expanded Recruitment Opportunities: FMN technology allows individuals with conditions like diabetes and irregular heart rates, who may have been previously ineligible to serve due to medical concerns, to now have the opportunity to join the military. This expands the pool of potential recruits and allows individuals with these conditions to fulfill their desire to serve.
Real-Time Health Monitoring: 26 Soldiers wear Bluetooth-enabled smartwatches and other wearables that continuously monitor vital health metrics such as blood pressure, oxygen saturation, heart rate, and activity tracking. Additionally, real-time insulin/glucose pumps and portable ECG/EKG devices can monitor diabetic and irregular heart rate states.
Secure Communication Infrastructure: The communications soldier 22 carries an Android device with satellite internet capability to communicate with the military's private network 20. The FMN app loaded on this device enables secure transmission of health data to the FMN server on the military network.
Unit Leader's Role: The unit leader 24 carries a ruggedized Android tablet or similar device with the FMN app loaded on it. They receive health data from soldiers 26 and transmit it through the communications soldier 22 to the satellite 20 for integration into the FMN server.
Monitoring Health Status: The FMN app provides real-time access to the soldiers' health, including monitoring diabetic and irregular heart rate states. It tracks and reports the condition of a soldier's health as they perform their duties, ensuring that any potential health issues are promptly addressed.
Alert System for Critical Situations: If a soldier experiences potential health issues such as low oxygen levels, prolonged elevated heart rate, or erratic glucose levels, the FMN app triggers alerts to both the unit leader and medical staff at the base in real-time. This allows for immediate intervention and medical assistance, ensuring the safety and well-being of the soldiers.
Secure Transport Technologies: FMN utilizes smart and secure transport technologies to ensure the confidentiality and integrity of the transmitted health data, even in high-stress situations. This ensures that crucial health information remains protected throughout the transmission process.
In summary, FMN technology enables potential soldiers with conditions like diabetes and irregular heart rates to serve in the military while ensuring that their health is closely monitored, and any potential health issues are addressed promptly. It enhances recruitment opportunities and contributes to the overall safety and well-being of soldiers on the battlefield.

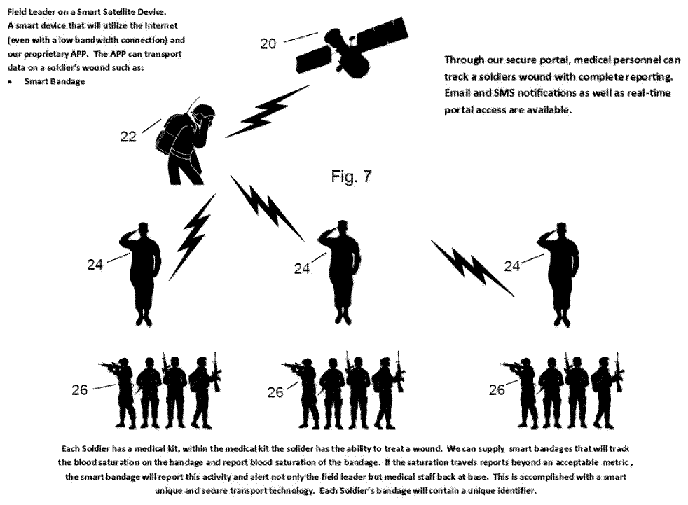

FIG. 7 The Family MedNet (FMN) technology provides crucial benefits for soldiers on the battlefield, especially in monitoring their wound status and responding to injuries. Here's how a potential soldier can benefit from FMN technology:
Real-Time Health Monitoring: 26 Soldiers wear Bluetooth-enabled smart bandages that continuously monitor vital health metrics such as wound status, bleeding, and other relevant health parameters.
Secure Communication Infrastructure: The communications soldier 22 carries an Android device with satellite internet capability to communicate with the military's private network 20. The FMN app loaded on this device enables secure transmission of health data to the FMN server on the military network.
Unit Leader's Role: The unit leader 24 carries a ruggedized Android tablet or similar device with the FMN app loaded on it. They receive health data from soldiers 26 wearing smart bandages and transmit it through the communications soldier 22 to the satellite 20 for integration into the FMN server.
Monitoring Wound Status: The FMN app provides real-time access to the soldiers' health, including monitoring the status of wounds and bleeding. It tracks and reports the condition of a soldier's health as they perform their duties, ensuring that any potential health issues related to injuries are promptly addressed.
Alert System for Wound Management: If a soldier experiences a potential health issue due to being wounded and requires bandaging, the FMN app triggers alerts to both the unit leader and medical staff at the base in real-time. This allows for immediate intervention and medical assistance, ensuring that wounds are properly addressed, and bleeding is controlled.
Secure Transport Technologies: FMN utilizes smart and secure transport technologies to ensure the confidentiality and integrity of the transmitted health data, even in high-stress situations. This ensures that crucial health information remains protected throughout the transmission process.
In summary, FMN technology plays a vital role in monitoring soldiers' health status, especially in response to injuries sustained on the battlefield. It enables timely intervention and medical support, contributing to better outcomes and increased safety for soldiers in challenging environments.

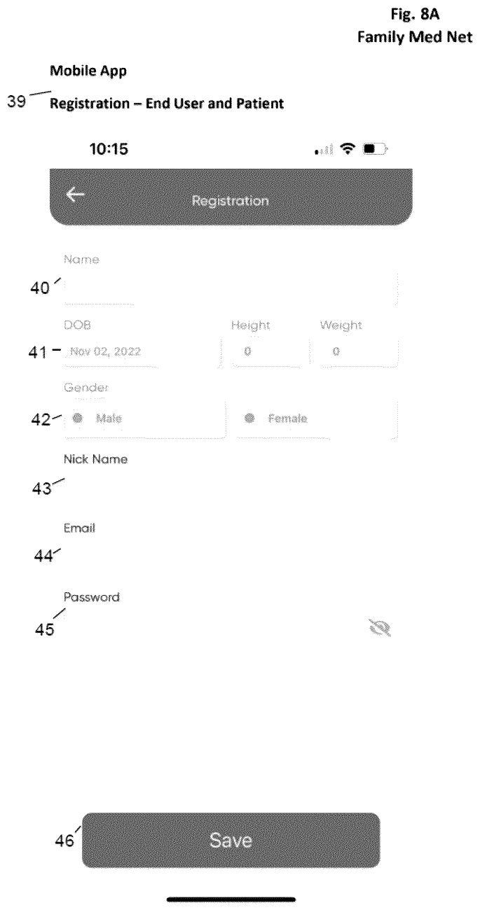

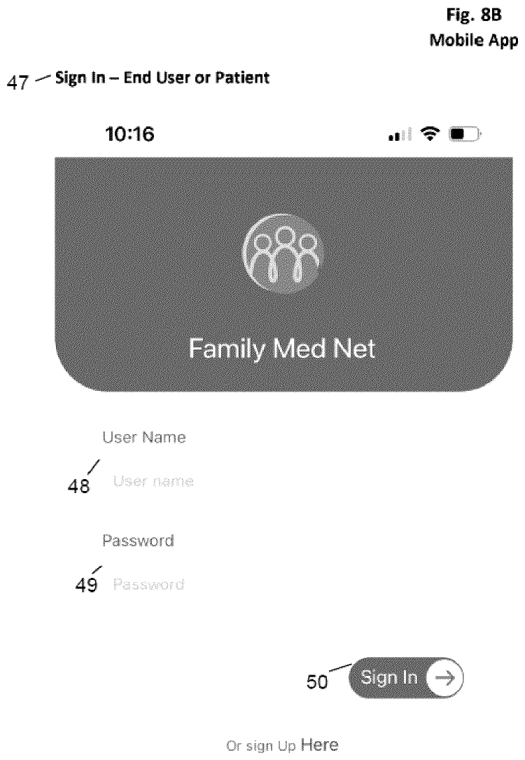

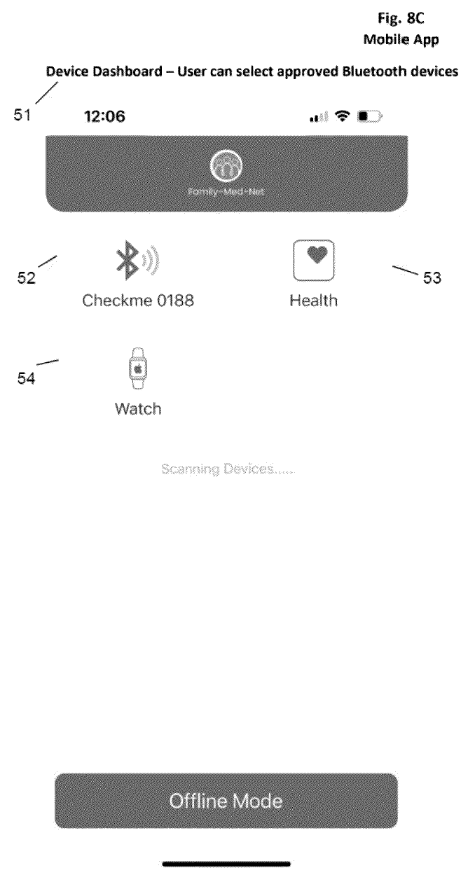

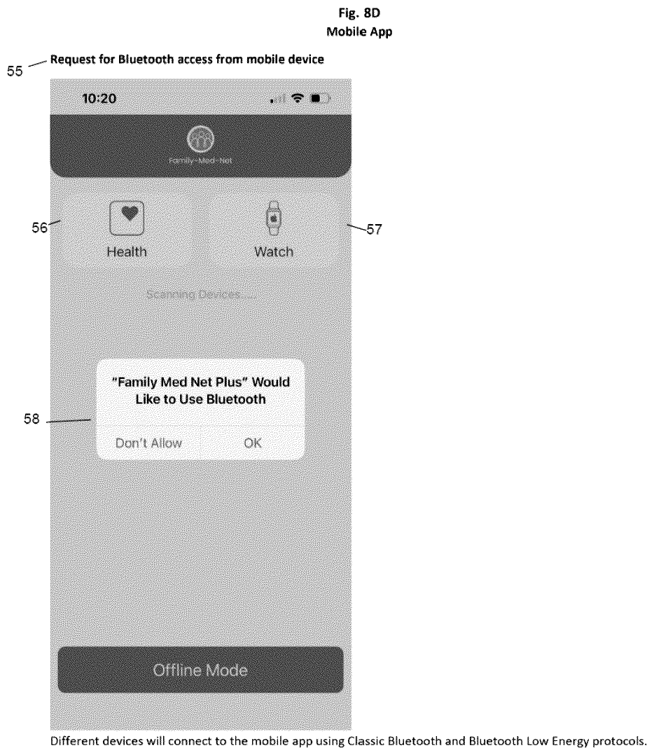

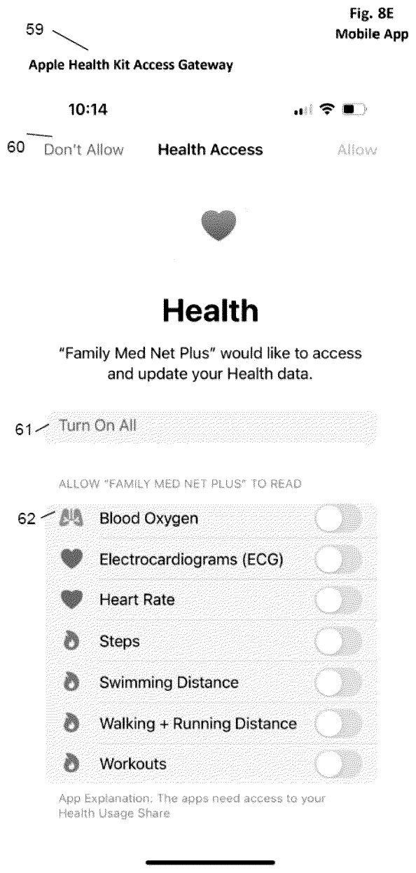

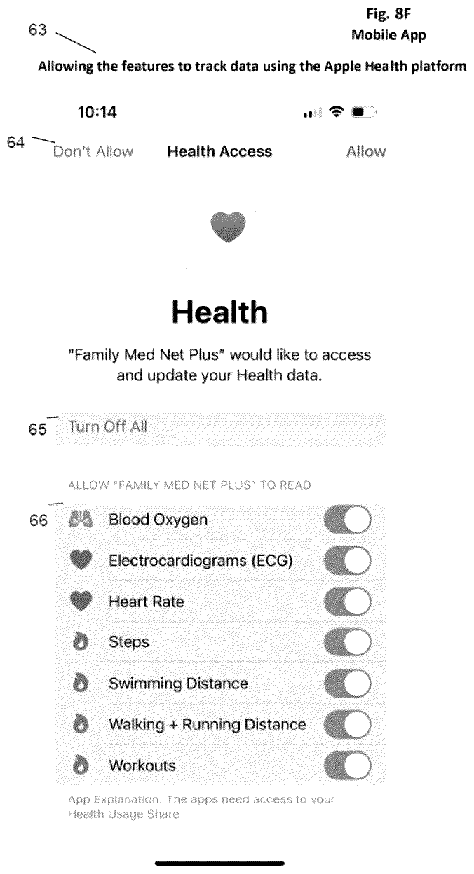

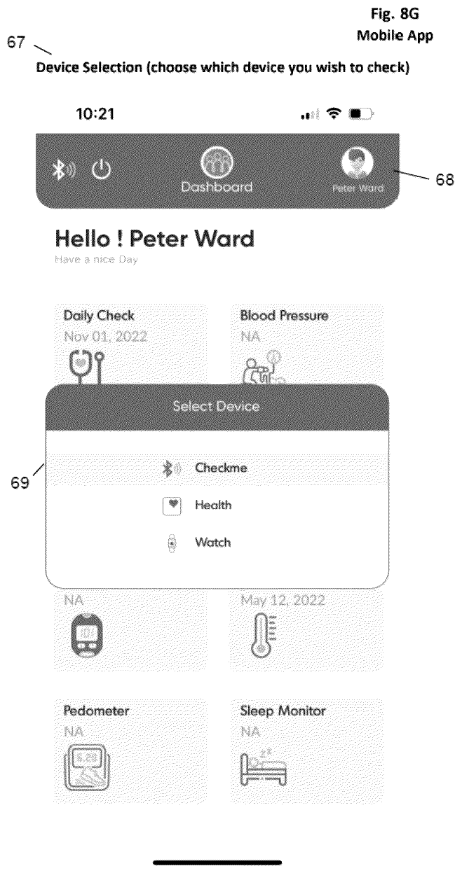

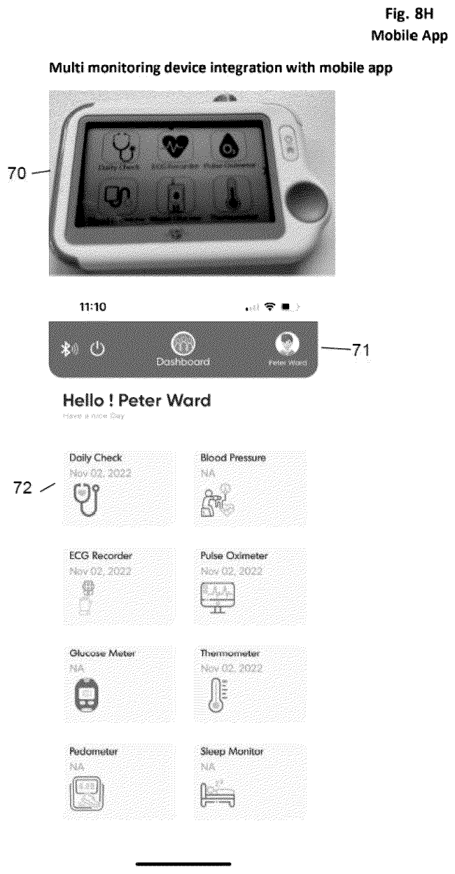

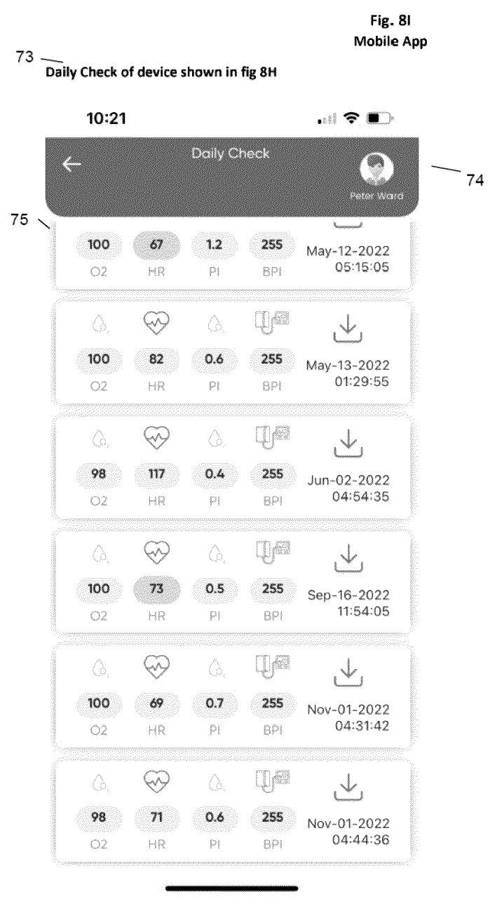

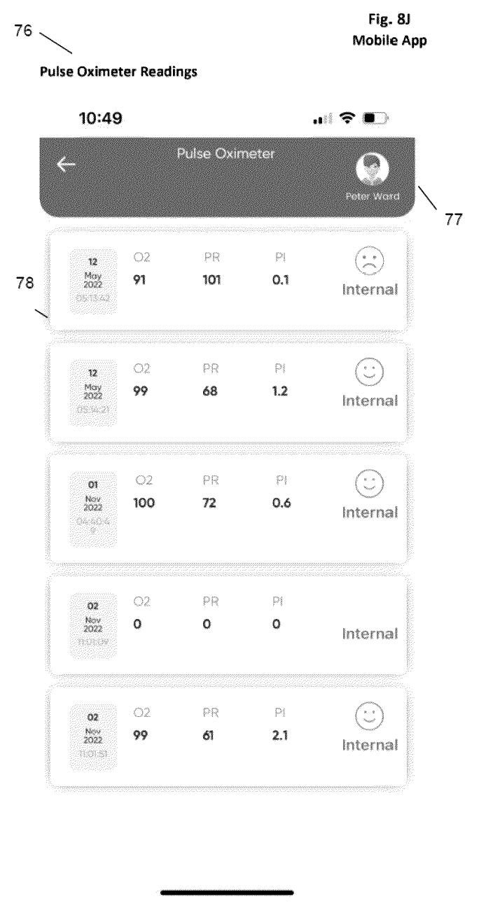

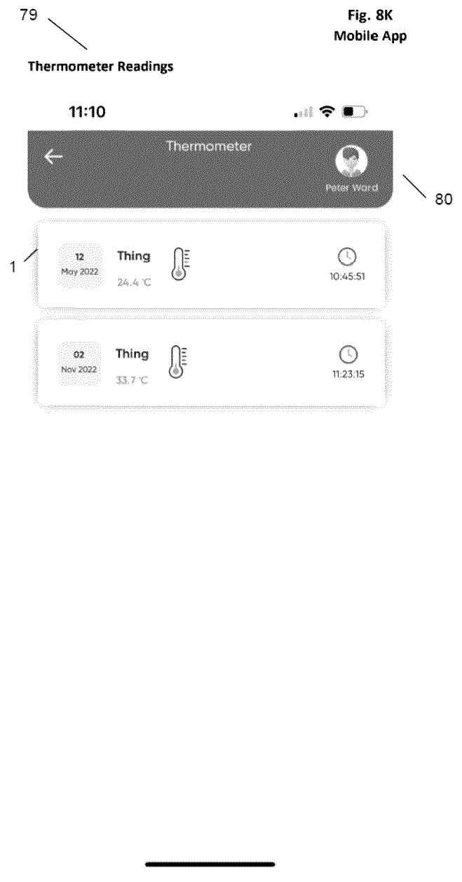

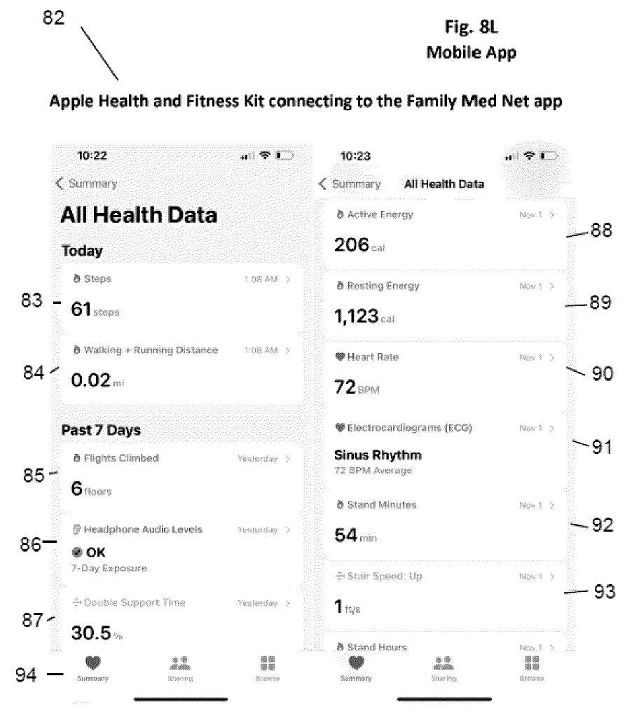

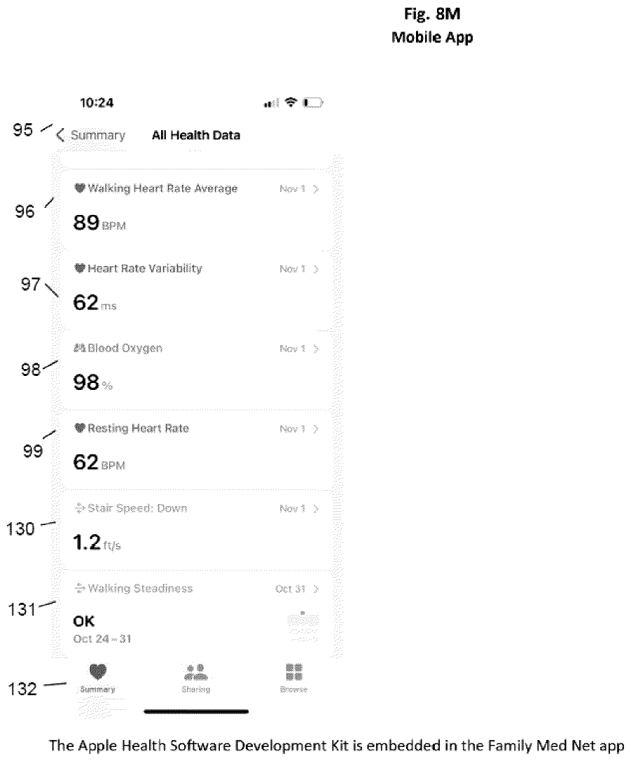

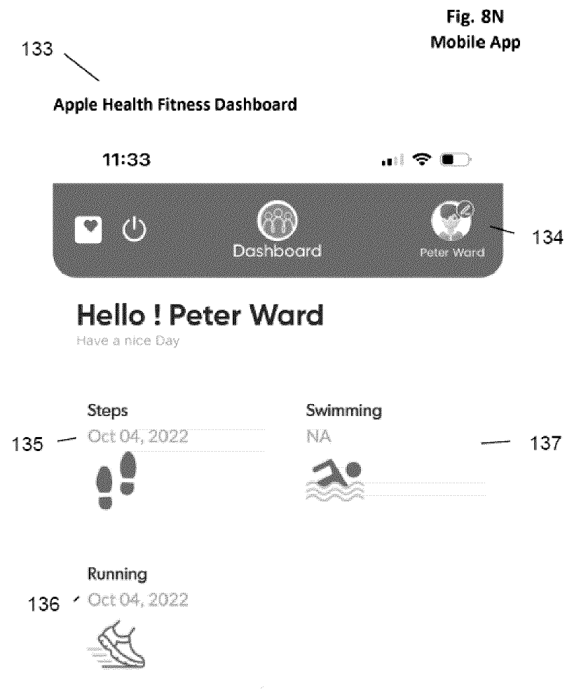

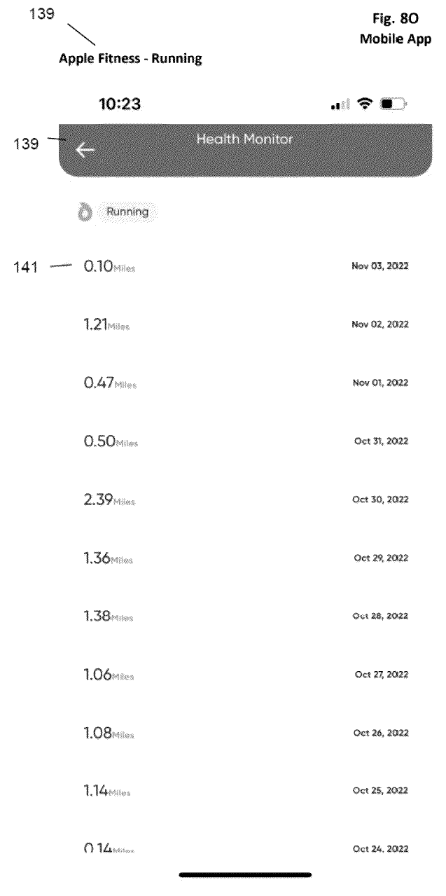

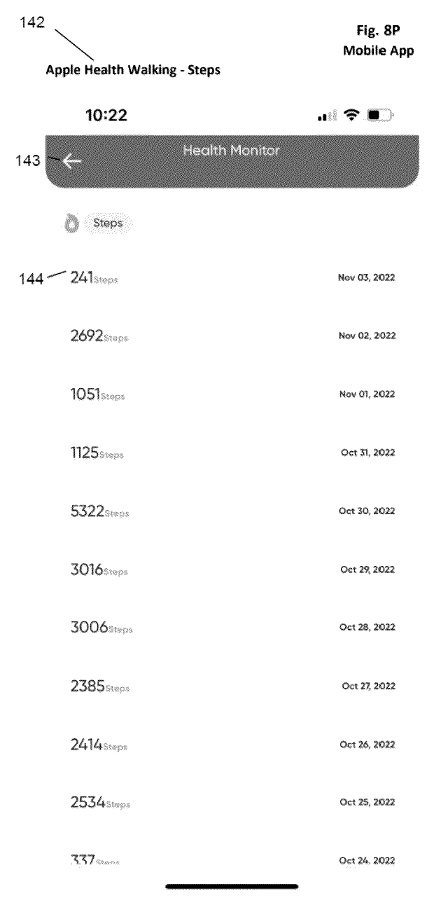

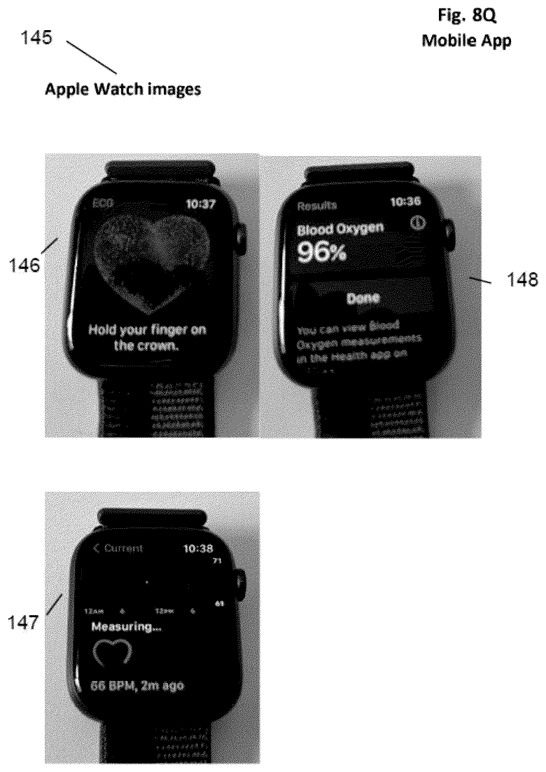

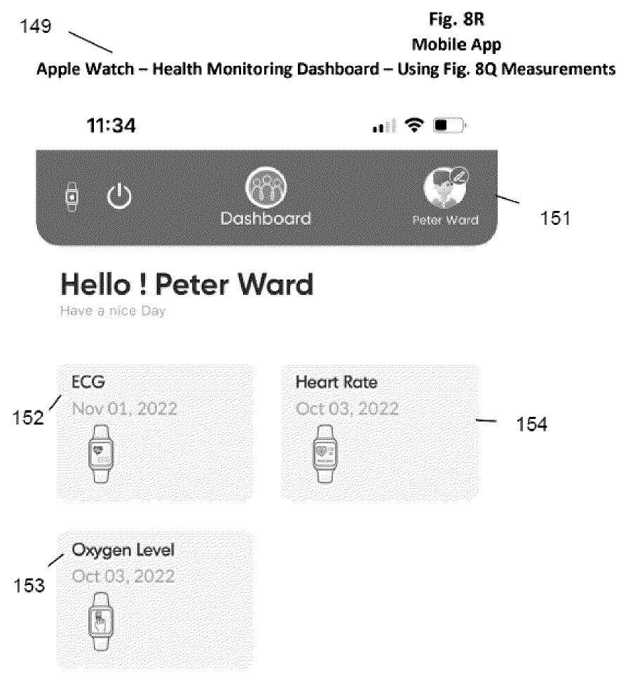

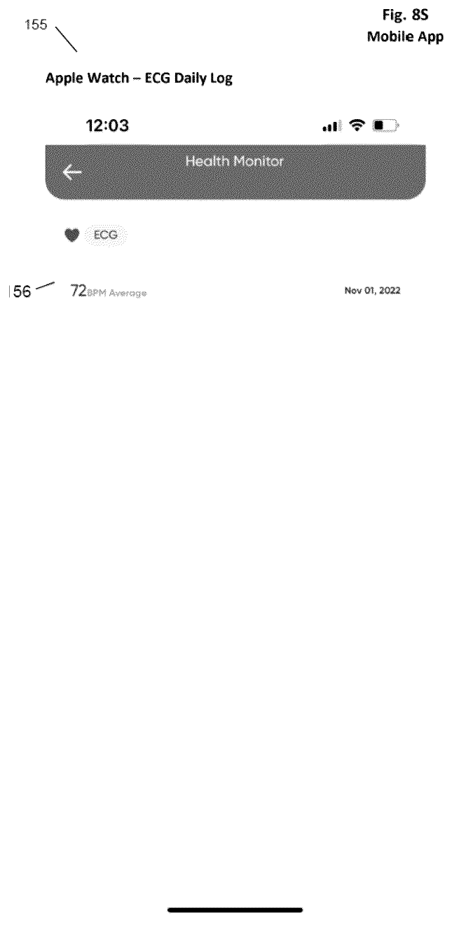

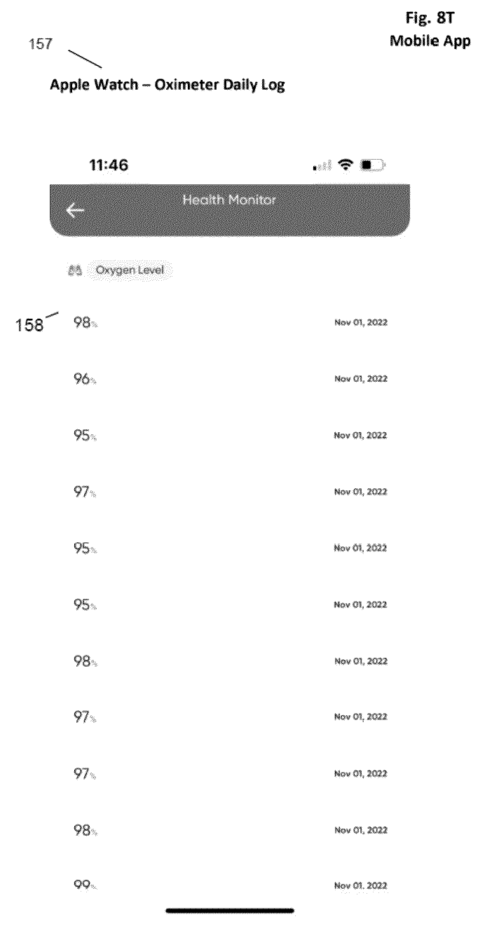

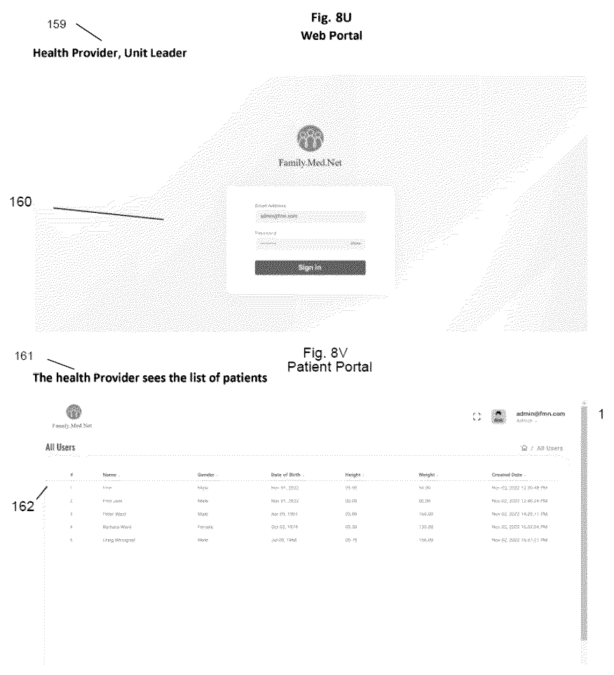

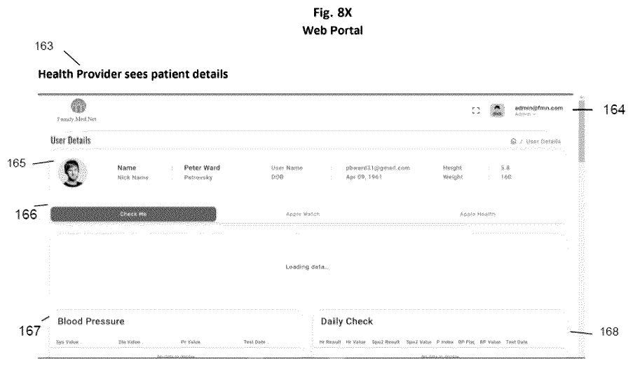

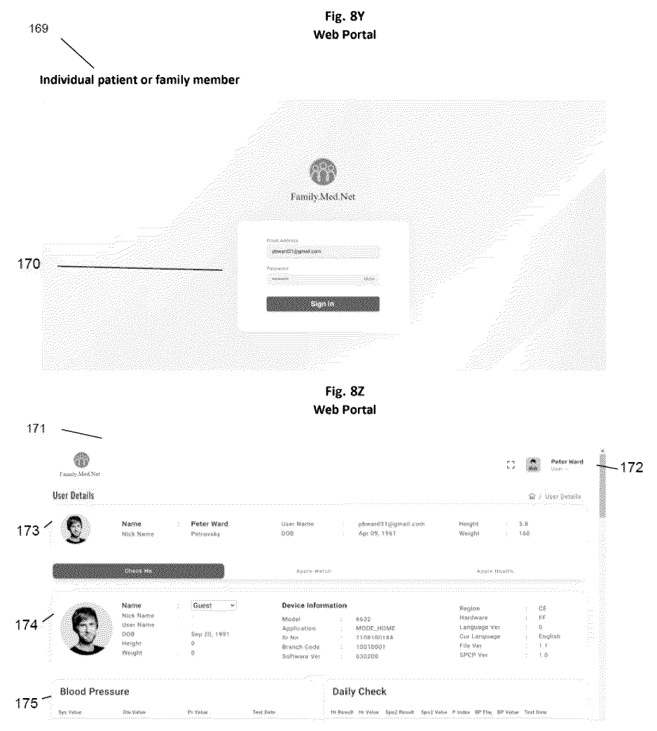

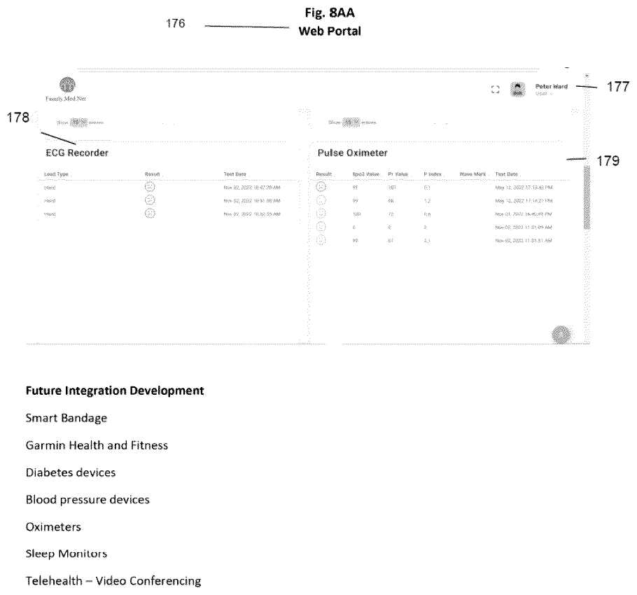

FIGS. 8A-8AA illustrate screen shots of a mobile app of the system.

FIG. 8A is a screenshot of a mobile app client registration page. This APP Screen demonstrates the collection of data for a monitored patient/end user to register for the Family MedNet Platform. This information can either be entered through this secure APP or through a secure registration site. The following data will need to be entered:
39 Registration Sign in Screen
40 Name
41 Date of Birth, Height, Weight
42 Gender (Radio Button Format)
43 Nickname
44 Email Address
45 Password
46 Save Button

FIG. 8B is a screenshot of a mobile app login page. This APP Screen demonstrates the login page for the APP (Same for Web Portal) for access to the FamilyMedNet Platform. We do provide another link to sign up. The following data will need to be entered:
47 User/Patient Sign In Screen
48 User Name (this will be the End User/Patient's Email Address
49 Password
50 Sign In Button

FIG. 8C is a screenshot of a mobile app device dashboard.

This APP screen demonstrates the device platform for a monitored patient on the Family MedNet Platform. The patient, family member and healthcare professional can easily add on approved Bluetooth Devices (Even Apple Health). This is associated with the unique registration (Pairing) of the Bluetooth Devices MAC address for the patient. Device references indicated by the following numbers:
51 Device Dashboard
52 Checkme (A Bluetooth multi monitoring device)
53 Apple Heath (Apple Iphone Health Log)
54 Apple Watch that can monitor heart Rate and other metrics

FIG. 8D is a screenshot of a mobile app request for Bluetooth access. This APP screen demonstrates acknowledgment to use the registered devices to start taking reading from the Bluetooth device for a monitored patient on the FamilyMedNet Platform. Device references to connect to indicated by the following numbers:
55 Request for Bluetooth Access
56 Apple Health
57 Apple Watch
58 The App request to use Bluetooth to connect to the device(s)

FIG. 8E is a screenshot of a mobile app Apple health kit access.

This APP screen demonstrates the integration with Apple Health for a monitored patient's metrics on the Family MedNet Platform. This helps to expand the Apple Health reporting including SMS to healthcare professionals. Sharing of this data is unlimited (Whereas Apple only can share with a limited number of contacts).

Apple Health metrics to include in share in monitoring indicated by the following numbers:
59 Apple Health Kit Gateway
60 Allow or not allow metrics
61 Turn on all metrics to monitor
62 The individual metrics you can use from the Apple Health Kit

FIG. 8F is a screenshot of a mobile app Apple health request to track health data. This APP screen demonstrates the integration with Apple Health for a monitored patient's metrics on the Family MedNet Platform. This helps to expand the Apple Health reporting including SMS to healthcare professionals. Sharing of this data is unlimited. You can toggle off and on the Health Care Access.

Apple Health metrics to include in tracking metrics indicated by the following numbers:
63 APP to Allow Apple Health Tracking
64 Allow or not allow metrics
65 Turn on all metrics to monitor
66 The individual metrics you can use from the Apple Health Kit

FIG. 8G is a screenshot of a mobile app device selection.

This APP screen demonstrates device platform for a monitored patient on the Family MedNet Platform. Patient, family member and or healthcare Professional can take readings of several devices currently simply by tapping on a device image shown in this figure. The following numbers indicate:
67 Device Selection to Check Screen
68 Shows Monitored User logged in
69 Choice of Which Device to monitor

FIG. 8H is a screenshot of a mobile app multi-monitoring device connection.

This APP screen demonstrates a supported multi-type device (Seen above the APP Screen) for a monitored patient on the Family MedNet Platform. Patient, family member and or healthcare Professional can take readings of several different health readings currently displayed. Just simply tap on the app screen shown in this figure to take a reading. The following numbers indicate:
70 A supported multi-use Bluetooth device
71 Shows Monitored User logged in.
72 Choice of Which feature you wish to monitor

FIG. 8I is a screenshot of a mobile app displaying the daily recorded health data including oxygen, heart rate, PI (perfusion index) and BPI (blood pressure).

This APP screen demonstrates the daily/weekly check on areas to be monitored thru the Multi-Type/Multi-Use Device from Figure “8H” for a monitored patient on the Family MedNet Platform. Each daily Check has the ability to share the readings with a download option Indicated by the down facing arrow on each reading to the far right. The following numbers indicate:
73 Daily Check Screen
74 Shows Monitored User logged in.
75 Examples of daily/weekly monitored readings

FIG. 8J is a screenshot of the Family MedNet mobile app displaying the daily recorded health data including oxygen, heart rate, PI (perfusion index) and BPI (blood pressure). This APP screen demonstrates the Pulse and Oximeter check for a monitored patient thru the Multi-Type Device from Figure “8H” for a monitored patient on the Family MedNet Platform. Notice the ‘Face” making it quicker to detect whether the reading was either favorable or not. These metrics can be set based on the patient's needs. The following numbers indicate:
76 Pulse Oximeter Reading Screen
77 Shows Monitored User logged in.
78 Examples of daily/weekly monitored readings of Oxygen levels

FIG. 8K is a screenshot of a mobile app displaying the daily recorded health data including temperature. This APP screen demonstrates the Thermometer check for a monitored patient thru the Multi-Type Device from Figure “8H” for a monitored patient on the Family MedNet Platform. Metrics can be set based on the patient's normal temperature for reporting. The following numbers indicate:
79 Thermometer Reading Screen
80 Shows Monitored User logged in.
81 Examples of daily/weekly monitored readings of body temperature

FIG. 8L is a screenshot of a mobile app displaying Apple health and fitness kit active summary connected to the system and real-time health data generated. This APP screen demonstrates the integration with Apple Health for a monitored patient's metrics on the Family MedNet Platform. This helps to expand Apple's Health reporting including SMS to healthcare professionals. Sharing of this data is unlimited. You can toggle off and on the Health Care Access (Seen in FIG. 8F). We have even incorporated steps, headphone audio levels. Imagine calling your parents to ask them why they are listening to music so loud. This could be an indication of hearing loss. The following numbers indicate:
82 Family MedNet App Displays Apple Health Kit Data Screen
83 Current day steps
84 Walking/running distance
85 Weekly stair flights climbed
86 Headphone Audio levels
87 Double Support Time (provides a measurement of the percentage of the gait cycle—from heel strike on one foot to heel strike on the contralateral foot—that a user spends on two feet)
88 Activity Energy
89 Resting Energy
90 Heart Rate
91 Sinus Rhythm
92 Stand Minutes
93 Stair Speed
94 Summary, Sharing and Browse a different screen

FIG. 8M is a screenshot of a mobile app displaying Apple health and fitness kit connected to the system and real-time health data generated. This APP screen demonstrates the integration with Apple Health for a monitored patient's metrics on the Family MedNet Platform. This helps to expand the Apple Health reporting on walking and exercise. This helps to expand the Apple Health reporting including SMS and emailing to family members and healthcare professionals. Sharing of this data is unlimited. The following numbers indicate:
95 24 Hour summary—Family MedNet App Displays Apple Health Kit Data Screen
96 Walking Heart rate average
97 Heart Rate Variability
98 Blood Oxygen
99 Resting Heart rate
130 Stair Speed down
131 Walking steadiness
132 Summary Sharing and Browse Bar

FIG. 8N is a screenshot of a mobile app displaying the Apple health and fitness kit dashboard. This APP screen demonstrates the integration with Apple Health for a monitored patient's metrics on the Family MedNet Platform. This helps to expand the Apple Health reporting on walking, swimming and exercise. This helps to expand the Apple Health reporting including SMS and email to family members, trainers, and healthcare professionals. Sharing of this data is unlimited. This is great for someone in training. The following numbers indicate:
133 FNM APP Display Apple Health Fitness Screen
134 Shows Monitored User logged in.
135 Steps tracked
136 Running tracked
137 Swimming tracked

FIG. 8O is a screenshot of a mobile app displaying Apple health and fitness kit connected to the system and real-time health data generated while running.

This APP screen demonstrates the integration with Apple Health for a monitored patient's metrics on the Family MedNet Platform. This helps to expand the Apple Health reporting on Running on a daily basis This helps to expand the Apple Health reporting including SMS and email to family members and trainers. Sharing of this data is unlimited.

The following numbers indicate:
138 FMN APP Displays Apple Fitness—Running Screen
139 Shows the Health Monitor screen header
141 Distance ran each day

FIG. 8P is a screenshot of a mobile app displaying Apple health and fitness kit connected to the system and real-time health data generated while walking.

This APP screen demonstrates the integration with Apple Health for a monitored patient's metrics on the Family MedNet Platform. This helps to expand the Apple Health reporting on walking on a daily basis. This helps to expand the Apple Health reporting including SMS and emailing to family members and trainers. Sharing of this data is unlimited.

The following numbers indicate:
142 FMN APP Displays Apple Fitness—Walking Screen
143 Shows the Health Monitor screen header
144 Steps reached per day

FIG. 8Q is a screenshot of Apple watches connected to the system.

This demonstrates the Apple Watch screen with Apple Health for a monitored patient's metrics on the Family MedNet Platform. This step shows the data for the collection of the Blood Oxygen levels to be displayed in Figure “8R” APP Screen. The following numbers indicate:
145 Apple Watch Images
146 ECG Reading
147 Heart Rate reading
148 Blood oxygen reading

FIG. 8R is a screenshot of a mobile app displaying health data generated by the Apple watch. This APP screen demonstrates the integration with Apple Health for a monitored patient's metrics on the Family MedNet Platform. This helps to expand the Apple Health reporting on Blood Oxygen on a daily basis pushed from the Apple Watch in Figure “8Q”. This helps to expand the Apple Health reporting including SMS and emailing to family members and trainers. Sharing of this data is unlimited. The following numbers indicate:
149 FMN APP Displays Apple Fitness—Walking Screen
151 Shows Monitored User logged in.
152 ECG Connection
153 Oxygen Level Connection
154 Heart Rate Connection

FIG. 8S is a screenshot of a mobile app displaying health data generated by the Apple watch including ECG. This APP screen demonstrates the integration with Apple Health for a monitored patient's metrics on the Family MedNet Platform. This helps to expand the Apple Health reporting on the ECG “Electrocardiogram” on a daily basis pushed from the Apple Watch

Figure “8Q”. This helps to expand the Apple Health reporting including SMS and emailing to family members and trainers. Sharing of this data is unlimited. The following numbers indicate:
155 FMN APP Displays Apple Health—ECG Screen
156 ECG Daily reading

FIG. 8T is a screenshot of a mobile app displaying health data generated by the Apple watch including the oxygen level. This APP screen demonstrates the integration with Apple Health for a monitored patient's metrics on the Family MedNet Platform. This helps to expand the Apple Health reporting on the Oxygen Levels on a daily basis. This helps to expand the Apple Health reporting including SMS and emailing to family members and trainers. Sharing of this data is unlimited. The following numbers indicate:
157 FMN APP Displays Apple Health—ECG Screen
158 Oxygen level tracking during training in a day

FIG. 8U is a screenshot of displaying a Web Portal for a health provider.

This webpage Login screen demonstrates the Healthcare provider login Family MedNet Platform. Notice the ability to monitor multiply patients. The following numbers indicate:
159 FMN Web Portal—Health Provider/Unit Leader Screen
160 Health Care provider login

FIG. 8V is a screenshot of the health providers portal displaying a list of patients for the health provider. The following webpage screen demonstrates a HealthCare Professionals list of monitored patient's portal on the Family MedNet Platform. This could be a White Label Platform where the Healthcare profession may Private Brand the pages for their practice. The following numbers indicate:
161 HealthCare Provider Screen to view All patients
162 List of All patients being monitored

FIG. 8X is a screenshot of a health care providers web portal displaying patient details for the health provider. The following webpage screen demonstrates a patient status portal on the Family MedNet Platform. All the Patients metrics will reside on this screen like Blood Pressure, Oxygen Levels, Glucose Levels for example. A picture of the patient can be uploaded for instant recognition. The following numbers indicate:
163 FMN Web Portal—Health Provider, Patient Details
164 Healthcare providers user ID/email address Displayed
165 Patients details
166 Devices being monitored
167 Blood Pressure
168 Daily Check

FIG. 8Y is a screenshot of the Family Mednet web portal login. The screen demonstrates the Patients or Family Member login for the Family MedNet Platform. The following numbers indicate:
169 FMN Web Portal—Patient or Family Member screen
170 Login for Patient of Family member

FIG. 8Z is a screenshot of a patient or family member's information that can be shared with a health care provider. This webpage screen demonstrates a parents status portal on the Family MedNet Platform. All the patient's metrics will reside on this screen like Blood Pressure, Oxygen Levels, Glucose Levels for example. A picture of the patient can be uploaded for instant recognition of the patient being monitored. The following numbers indicate:
171 FMN Web Patients Portal
172 Shows Monitored User logged in
173 Patient's details
174 Devices monitoring patient
175 Device Readings

FIG. 8AA is a screenshot of more device readings from the bottom of from The following numbers indicate:
176 FMN Web Patients Portal
177 Shows Monitored User logged in.
178 ECG Recorder reading
179 Pulse Oximeter Reading

While the invention has been described with reference to particular embodiments, it will be understood by those skilled in the art that various changes may be made and equivalents may be substituted for elements thereof without departing from the scope of the invention. In addition, many modifications may be made to adapt a particular situation or material to the teachings of the invention without departing from the scope of the invention. Therefore, it is intended that the invention not be limited to the particular embodiments disclosed as the best mode contemplated for carrying out this invention, but that the invention will include all embodiments falling within the scope and spirit of the appended claims.

### REFERENCE NUMBERS

1 Family Mednet (FMN) Approved Bluetooth Medical Internet of Things Device(s)
2 Bluetooth connection
3 Family MedNet (FMN) APP and Unique Method of Bluetooth MAC identification
4 Secure Socket Layer (SSL) Data Upload
5 Family MedNet (FMN) Front End Web Server
6 SSL Data Passthru
7 FMN Webserver with Alerting for Patients
8 SSL Data Passthru
9 SQL Server (Database) Places Patient IoT MAC Address into record
10 Bluetooth Medical Devices
11 Family Mednet (FMN) App
12 Family Mednet Secure Cloud Server
13 Monitoring and Care
20 Satellite Network
22 Communications Soldier
24 Unit leader
26 Soldiers
39 Registration Sign in Screen
40 Name
41 Date of Birth, Height, Weight
42 Gender (Radio Button Format)
43 Nickname
44 Email Address
45 Password
46 Save Button
47 User/Patient Sign In Screen
48 User Name (this will be the End User/Patient's Email Address
49 Password
50 Sign In Button
51 Device Dashboard
52 Checkme (A Bluetooth multi monitoring device)
53 Apple Heath (Apple Iphone Health Log)
54 Apple Watch that can monitor heart Rate and other metrics
55 Request for Bluetooth Access
56 Apple Health
57 Apple Watch
58 The App request to use Bluetooth to connect to the device(s)
59 Apple Health Kit Gateway
60 Allow or not allow metrics
61 Turn on all metrics to monitor
62 The individual metrics you can use from the Apple Health Kit
63 APP to Allow Apple Health Tracking
64 Allow or not allow metrics
65 Turn on all metrics to monitor
66 The individual metrics you can use from the Apple Health Kit
67 Device Selection to Check Screen
68 Shows Monitored User logged in
69 Choice of Which Device to monitor
70 A supported multi-use Bluetooth device
71 Shows Monitored User logged in.
72 Choice of Which feature you wish to monitor
73 Daily Check Screen
74 Shows Monitored User logged in.
75 Examples of daily/weekly monitored readings
76 Pulse Oximeter Reading Screen
77 Shows Monitored User logged in.
78 Examples of daily/weekly monitored readings of Oxygen levels
79 Thermometer Reading Screen
80 Shows Monitored User logged in.
81 Examples of daily/weekly monitored readings of body temperature
82 Family MedNet App Displays Apple Health Kit Data Screen
83 Current day steps
84 Walking/running distance
85 Weekly stair flights climbed
86 Headphone Audio levels
87 Double Support Time (provides a measurement of the percentage of the gait cycle—from heel strike on one foot to heel strike on the contralateral foot—that a user spends on two feet)
88 Activity Energy
89 Resting Energy
90 Heart Rate
91 Sinus Rhythm
92 Stand Minutes
93 Stair Speed
94 Summary, Sharing and Browse a different screen
95 24 Hour summary—Family MedNet App Displays Apple Health Kit Data Screen
96 Walking Heart rate average
97 Heart Rate Variability
98 Blood Oxygen
99 Resting Heart rate
130 Stair Speed down
131 Walking steadiness
132 Summary Sharing and Browse Bar
133 FNM APP Display Apple Health Fitness Screen
134 Shows Monitored User logged in.
135 Steps tracked
136 Running tracked
137 Swimming tracked
138 FMN APP Displays Apple Fitness—Running Screen
139 Shows the Health Monitor screen header
141 Distance ran each day
142 FMN APP Displays Apple Fitness—Walking Screen
143 Shows the Health Monitor screen header
144 Steps reached per day
145 Apple Watch Images
146 ECG Reading
147 Heart Rate reading
148 Blood oxygen reading
149 FMN APP Displays Apple Fitness—Walking Screen
151 Shows Monitored User logged in.
152 ECG Connection
153 Oxygen Level Connection
154 Heart Rate Connection
155 FMN APP Displays Apple Health—ECG Screen
156 ECG Daily reading
157 FMN APP Displays Apple Health—ECG Screen
158 Oxygen level tracking during training in a day
159 FMN Web Portal—Health Provider/Unit Leader Screen
160 Health Care provider login
161 HealthCare Provider Screen to view All patients
162 List of All patients being monitored
163 FMN Web Portal—Health Provider, Patient Details
164 Healthcare providers user ID/email address Displayed
165 Patients details
166 Devices being monitored
167 Blood Pressure
168 Daily Check
169 FMN Web Portal—Patient or Family Member screen
170 Login for Patient of Family member
171 FMN Web Patients Portal
172 Shows Monitored User logged in
173 Patient's details
174 Devices monitoring patient
175 Device Readings
176 FMN Web Patients Portal
177 Shows Monitored User logged in.
178 ECG Recorder reading
179 Pulse Oximeter Reading
100 System
120 User Interface Device
140 Network
140 Server

We claim:

1. A system for monitoring in real time health data of an individual comprising:
a cloud-based server connected to a network, the cloud-based server comprising a tangible, non-volatile memory;
at least one medical device wirelessly connected to the network, wherein the at least one medical device is configured to generate health data from the individual, and the at least one medical device having a unique MAC address that is associated with the individual; and
at least one user interface device connected to the network, wherein the server is configured to store the health data and to allow the at least one user interface device to view the health data in real time and associate the health data with the individual based on the unique MAC address.

2. The system according to claim 1, wherein the MAC address is a Bluetooth MAC address.

3. The system according to claim 2, wherein the at least one device is a Bluetooth enabled thermometer and the health data includes temperature readings from the thermometer.

4. The system according to claim 2, wherein the at least one device is a Bluetooth enabled blood pressure monitor and the health data includes blood pressure readings from the blood pressure monitor.

5. The system according to claim 2, wherein the at least one device is a Bluetooth enabled pill box and the health data includes a pill dispensed from the pill box.

6. The system according to claim 2, wherein the at least one device is a Bluetooth enabled urinalysis device and the health data includes urinalysis readings from the urinalysis device.

7. The system according to claim 2, wherein the at least one device is a Bluetooth enabled blood glucose device and the health data includes blood glucose readings from the blood glucose device.

8. The system according to claim 2, wherein the at least one device is a Bluetooth enabled heart monitoring device and the health data includes heart readings from the heart monitoring device.

9. The system according to claim 2, wherein the at least one device is a Bluetooth enabled bandage and the health data includes bandage readings from the bandage.

10. The system according to claim 2, wherein the at least one device is a Bluetooth enabled smart watch and the health data includes watch readings from the smart watch.

11. A method of monitoring health data of an individual in real time comprising:
providing a system comprising a cloud-based server connected to a network, the cloud-based server comprising a tangible, non-volatile memory;
at least one medical device wirelessly connected to the network, wherein the at least one medical device is configured to generate health data from the individual, and the at least one medical device having a unique MAC address that is associated with the individual; and
at least one user interface device connected to the network, wherein the server is configured to store the health data and to allow the at least one user interface device to view the health data in real time and associate the health data with the individual based on the unique MAC address
using the user interface device to remotely monitor the health data of the individual in real time; and
storing the health data in the tangible, non-volatile memory.

12. The method according to claim 11, wherein the MAC address is a Bluetooth MAC address.

13. The method according to claim 12, wherein the at least one device is a Bluetooth enabled thermometer and the health data includes temperature readings from the thermometer.

14. The method according to claim 12, wherein the at least one device is a Bluetooth enabled blood pressure monitor and the health data includes blood pressure readings from the blood pressure monitor.

15. The method according to claim 12, wherein the at least one device is a Bluetooth enabled pill box and the health data includes a pill dispensed from the pill box.

16. The method according to claim 12, wherein the at least one device is a Bluetooth enabled urinalysis device and the health data includes urinalysis readings from the urinalysis device.

17. The method according to claim 12, wherein the at least one device is a Bluetooth enabled blood glucose device and the health data includes blood glucose readings from the blood glucose device.

18. The method according to claim 12, wherein the at least one device is a Bluetooth enabled heart monitoring device and the health data includes heart readings from the heart monitoring device.

19. The method according to claim 12, wherein the at least one device is a Bluetooth enabled bandage and the health data includes bandage readings from the bandage.

20. The method according to claim 12, wherein the at least one device is a Bluetooth enabled smart watch and the health data includes watch readings from the smart watch.

21. The method according to claim 12, wherein the at least one device is a Bluetooth enabled smart watch and the health data includes watch readings from the smart watch.

22. The method according to claim 12, wherein the at least one device is a Bluetooth enabled blood glucose device and the health data includes blood glucose readings from the blood glucose device, and tracking real time the blood glucose level, determining a level of blood glucose, and dispensing insulin to reduce the level of blood glucose on the patient.

23. The method according to claim 12, comprising importing the health data into an EMR system.

24. The method according to claim 12, comprising monitoring a plurality of patients wherein each patient has an associated at least one medical device, and each patient has an associated unique Bluetooth MAC address.

25. The method according to claim 24, wherein the patients are monitored in a nursing home.

26. The method according to claim 24, wherein the patients are monitored in a doctor's office through White Label.

27. The method according to claim 24, wherein the patients are monitored remotely through White Label.

28. The method according to claim 12, comprising monitoring a plurality of soldiers wherein each soldier has an associated at least one medical device, and each soldier has an associated unique Bluetooth MAC address.

29. The method according to claim 28, wherein the at least one medical device detects a gunshot in a soldier.
